(19) United States
(12) Patent Application Publication
(10) Pub. No.: US 2017/0056309 A1
(43) Pub. Date: Mar. 2, 2017

(54) LUMINATE FACE MASK

(71) Applicant: Jan Marini Skin Research, San Jose, CA (US)

(72) Inventors: Jan L. Marini, San Jose, CA (US); Subhash J. Saxena, Ringoes, NJ (US)

(21) Appl. No.: 14/835,514

(22) Filed: Aug. 25, 2015

(51) Int. Cl. A61K 8/60; A61K 8/63; A61K 8/02; A61K 8/92; A61K 8/26; A61Q 19/02; A61K 8/49; A61K 8/365

(52) U.S. Cl. CPC A61K 8/602; A61K 8/498; A61K 8/63; A61K 8/365; A61K 8/922; A61K 8/26; A61Q 19/02; A61K 8/0212; A61K 2800/74; A61K 2800/596; A61K 2800/42

(57) ABSTRACT

The present invention features novel cosmetic skin care compositions for improving the appearance of skin, particularly the reducing the appearance of age spots and discoloration.

### BACKGROUND OF THE INVENTION

Researchers have examined aspects of human faces that affect perceptions of health, attractiveness, and age. Recently, several studies have focused on the contributions of skin color and texture to apparent facial health and attractiveness. The homogeneity and distribution of skin color across the face contributes to perceptions of health, age, and attractiveness of human faces. Ratings of facial attractiveness correlated with measures of skin color homogeneity, such that more homogeneous skin color distribution was more attractive. More homogeneous chromophore (melanin and hemoglobin) distribution across the skin also correlates positively with ratings of health, attractiveness, and youthfulness, while the facial color distribution of younger women is perceived as younger, healthier, and more attractive than that of older women.

Further, increased contrast between the luminance of the facial features and the rest of the facial skin has been shown to enhance the attractiveness and femininity of female faces. Evidence suggests that skin color and texture themselves contribute to the attractiveness of faces independently of shape, and when color distribution across the whole face cannot be viewed.

Cosmetic products that improve skin tone, texture and luminosity are of great interest, and can provide a complement to skin care routines to combat pigmentation.

### SUMMARY OF THE INVENTION

The present invention provides cosmetic formulations for improving the appearance of the skin, including the facial area. The compositions of the invention are designed for use as a mask to improve skin tone, texture and luminosity by providing multiple ingredients to address the major factors that contribute to the appearance of aging skin for immediate lightening and evening of skin tone. The compositions of the invention are non-drying, paraben-free and fragrance-free.

The compositions of the invention include lightening agents to progressively reduce the appearance of spots and discoloration, exfoliating agents to remove dull, dry skin for a more hydrated, radiant glow; and moisturizing and calming agents reduce the appearance of redness. An occlusive clay-base increases penetration, reduces oily skin, and rinses cleanly from the skin, removing dirt and debris. The composition is topically administered as a mask, and left on the skin for a period of time sufficient to accomplish the desired effect before rinsing off. In some embodiments the composition is administered from about one to three times per week.

According to the first aspect of the invention, there is provided a cosmetic composition comprising a specific and efficacious blend of agents, including skin brightening agents dimethylmethoxy chromanyl palmitate, alpha arbutin and dipotassium glycyrrhizate. Exfoliating agents lactic acid and mandelic acid are also included. The compositions comprise a blend of tea extracts as a source of anti-oxidants and calming agents, comprising extracts of Camellia sinensis and C. oleifera, and Aspalathus linearis extract. The formulation base comprises kaolin.

In the second aspect of the invention, a method is provided for improving the appearance of the skin, in particular to improve skin tone, texture and luminosity, the method comprising applying topically a cosmetic mask composition comprising: skin brightening agents dimethylmethoxy chromanyl palmitate, alpha arbutin and dipotassium glycyrrhizate; exfoliating agents lactic acid and mandelic acid; a blend of tea extracts comprising extracts of Camellia sinensis, C. oleifera, and Aspalathus linearis in a kaolin clay base.

### DETAILED DESCRIPTION OF THE EMBODIMENTS

Topical compositions are provided for improving the appearance of the skin, particularly in lightening and evening skin tone. The compositions of the invention include a cosmetically acceptable vehicle to act as a diluent, dispersant or carrier for the active agents, so as to facilitate distribution and uptake when the composition is applied as a skin mask, which is rinsed off after a period of time. Vehicles other than or in addition to kaolin and water can include liquid or solid emollients, solvents, humectants, thickeners and powders. The cosmetically acceptable vehicle will usually form 5% to 99.9%, preferably from 25% to 80%, about 40% to 60%, by weight of the composition, and can, in the absence of other cosmetic adjuncts, form the balance of the composition.

The compositions may be in the form of an aqueous clay dispersion. These compositions are formulated according to the usual techniques as are well known to this art. The compositions of the invention may further comprise cosmetically useful agents and excipients, e.g. glycerin, cetyl alcohol, capric triglyceride, glyceryl stearate, PEG-100 stearate, steareth-20, steareth-2, cyclopentasiloxane, phenoxyethanol, lecithin, tocopherol, aloe vera, etc. each at a concentration of from about 0.1% to about 10% by weight, usually from about 0.5% to about 5%, and may be present at a concentration of from about 0.5%, ₁%_{, 2}%_{, 3}%_{, 4}%_{, 5}%_{,} etc.

### Components of the Cosmetic Compositions

The compositions of the invention comprise a specific blend of therapeutic agents. Dimethylmethoxy chromanyl palmitate (CAS 1105025-85-1) may be present in the composition at a final concentration of from about 0.025% to about 1% by weight, usually about 0.05 to 0.5% and may be present at about 0.1% by weight. Dimethylmethoxy chromanyl palmitate (INCI name) is a novel skin brightener that has been tested for efficacy and safety with satisfactory results. The agent does not have any of the side effects that are normally associated with depigmenting agents, presents good efficacy in vitro and in vivo and is unique in that, unlike all other skin brighteners, it has a photoprotective effect.

Lightening agents in the formulation include alpha arbutin at a concentration of from about 0.05% to about 1%, usually from about 0.1% to about 0.5%, and may be about 0.3% by weight. Alpha-Arbutin (4-Hydroxyphenyl-a-D-glucopyranoside) is a functional active ingredient for skin lightening, CAS# 84380-01-8. Alpha-Arbutin blocks epidermal melanin biosynthesis by inhibiting enzymatic oxidation of Tyrosine and Dopa.

Dipotassium glycyrrhizate may be present in the composition at a concentration of from about 0.05% to about 1% by weight, usually about 0.1% to about 1%, and may be present at about 0.5% by weight. Dipotassium glycyrrhizate is a licorice root extract with skin lightening properties. Dipotassium glycyrrhizate is known in the art and commercially available under CAS #68797-35-3 from various suppliers.

Exfoliating agents in the composition include alpha hydroxy acids (AHA), including without limitation lactic acid and mandelic acid. Lactic acid, CAS 79-33-4, (S)-2-Hydroxypropionic acid, is present at a concentration of from about 1% to about 10%, usually about 2.5% to about 7.5%, and may be around about 5% by weight. Mandelic acid, CAS 90-64-2, α-Hydroxyphenylacetic acid, is present at a concentration of from about 1% to about 10%, usually about 2.5% to about 7.5%, and may be around about 5% by weight. AHAs as used in cosmetics function as exfoliants, acting on the surface of the skin by removing dead surface cells, thereby improving the appearance of the skin. In addition, lactic acid functions as a humectant-skin conditioning agent. AHAs also function as pH adjusters.

A blend of tea extracts in the composition provide anti-oxidants and skin calming agents. Tea extracts include extracts of Camellia sinensis, Camellia oleifera and Aspalathus linearis. Tea extracts contain polyphenols with antioxidant and anti-inflammatory properties. Epigallocatechin-3-gallate (EGCG), an extract of tea, can prevent collagen breakdown and reduce UV damage to skin. Camellia sinensis extract (CAS 84650-60-2) may be present at a concentration of from about 0.05% to about 1%, usually from about 0.1% to about 0.5%, and may be about 0.2% by weight. Camellia oleifera extract (CAS 94333-93-4) may be present at a concentration of from about 0.05% to about 1%, usually from about 0.1% to about 0.5%, and may be about 0.2% by weight. Aspalathus linearis may be present at a concentration of from about 0.05% to about 1%, usually from about 0.1% to about 0.5%, and may be about 0.2% by weight.

The cosmetic base of the formulation contains kaolin (0AS1332-58-7), at a concentration by weight of from about 5% to about 15% by weight, usually from about 7.5% to about 12.5%, and may be present at around 10% by weight. Kaolin is a naturally occurring soft clay, that helps to cleanse and exfoliate dead skin cells and debris from the surface of the skin.

The compositions of the invention may optionally comprise other skin benefit materials. These include estradiol; progesterone; pregnanalone; coenzyme Q10; methylsolanomethane (MSM); copper peptide (copper extract); plankton extract (phytosome); kojic acid; broparoestrol; estrone; adrostenedione; androstanediols; hydroquinone; isoflavones, etc. The steroids will generally be present at a concentration of less than about 5% or about 10% of the total by weight of the composition, while the other skin benefit materials may be present at higher levels, for example as much as about 10 to about 15%.

The amounts of cosmetic or dermatological auxiliaries and additives and perfume to be used in each case can easily be determined by simple exploratory experiments by the person skilled in the art, depending on the nature of the product in question. Preferably the compositions of the invention are fragrance free and paraben-free.

### Cosmetically Acceptable Vehicle

The compositions of the invention include a cosmetically acceptable vehicle to act as a diluent, dispersant or carrier for the active agents, so as to facilitate distribution and uptake when the composition is applied to the skin. Vehicles other than or in addition to water can include liquid or solid emollients, solvents, humectants, thickeners and powders.

The compositions of the invention include a cosmetically acceptable vehicle to act as a diluent, dispersant or carrier for the active agents, so as to facilitate distribution and uptake when the composition is applied as a skin mask, which is rinsed off after a period of time. Vehicles other than or in addition to kaolin and water can include liquid or solid emollients, solvents, humectants, thickeners and powders. The cosmetically acceptable vehicle will usually form 5% to 99.9%, preferably from 25% to 80%, about 40% to 60%, by weight of the composition, and can, in the absence of other cosmetic adjuncts, form the balance of the composition.

The compositions of the invention may also contain additives and adjuvants which are conventional in the cosmetic, pharmaceutical or dermatological field, such as hydrophilic or lipophilic gelling agents, hydrophilic or lipophilic active agents, preservatives, antioxidants, solvents, fragrances, fillers, bactericides, odor absorbers and dyestuffs or colorants. The amounts of these various additives and adjuvants are those conventionally used in the field, and, for example, range from 0.01% to 10% of the total weight of the composition. Depending on their nature, these additives and adjuvants may be introduced into the fatty phase or into the aqueous phase.

Exemplary oils which may be used according to this invention include mineral oils (liquid petrolatum), plant oils (liquid fraction of karite butter, sunflower oil), animal oils (perhydrosqualen(e), synthetic oils (purcellin oil), silicone oils (cyclomethicone) and fluoro oils (perfluoropolyethers). Fatty alcohols, fatty acids (stearic acid) and waxes (paraffin wax, carnauba wax and beeswax) may also be used as fats.

Emulsifiers which may be used include glyceryl stearate, polysorbate 60, PEG-6/PEG-32/glycol stearate mixture, etc. Solvents which may be used include the lower alcohols, in particular ethanol and isopropanol, and propylene glycol.

Hydrophilic gelling agents include carboxyvinyl polymers (carbomer), acrylic copolymers such as acrylate/alkylacrylate copolymers, polyacrylamides, polysaccharides, such as hydroxypropylcellulose, natural gums and clays, and, as lipophilic gelling agents, representative are the modified clays such as bentones, fatty acid metal salts such as aluminum stearates and hydrophobic silica, or ethylcellulose and polyethylene.

An oil or oily material may be present, together with an emollient to provide either a water-in-oil emulsion or an oil-in-water emulsion, depending largely on the average hydrophilic-lipophilic balance (HLB) of the emollient employed. Levels of such emollients may range from about 0.5% to about 50%, preferably between about 5% and 30% by weight of the total composition. Emollients may be classified under such general chemical categories as esters, fatty acids and alcohols, polyols and hydrocarbons.

Esters may be mono- or di-esters. Acceptable examples of fatty di-esters include dibutyl adipate, diethyl sebacate, diisopropyl dimerate, and dioctyl succinate. Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, oleyl eurcate and stearyl oleate. Preferred esters include coco-caprylate/caprate (a blend of coco-caprylate and coco-caprate), propylene glycol myristyl ether acetate, diisopropyl adipate and cetyl octanoate.

Suitable fatty alcohols and acids include those compounds having from 10 to 20 carbon atoms. Especially preferred are such compounds such as cetyl, myristyl, palmitic and stearyl alcohols and acids.

Among the polyols which may serve as emollients are linear and branched chain alkyl polyhydroxyl compounds. For example, propylene glycol, sorbitol and glycerin are preferred. Also useful may be polymeric polyols such as polypropylene glycol and polyethylene glycol. Butylene and propylene glycol are also especially preferred as penetration enhancers.

Exemplary hydrocarbons which may serve as emollients are those having hydrocarbon chains anywhere from 12 to 30 carbon atoms. Specific examples include mineral oil, petroleum jelly, squalene and isoparaffins.

Another category of functional ingredients within the cosmetic compositions of the present invention are thickeners. A thickener will usually be present in amounts anywhere from 0.1 to 20% by weight, preferably from about 0.5% to 10% by weight of the composition. Exemplary thickeners are cross-linked polyacrylate materials available under the trademark Carbopol. Gums may be employed such as xanthan, carrageenan, gelatin, karaya, pectin and locust beans gum. Under certain circumstances the thickening function may be accomplished by a material also serving as a silicone or emollient. For instance, silicone gums in excess of 10 centistokes and esters such as glycerol stearate have dual functionality.

Powders may be incorporated into the cosmetic composition of the invention. These powders include chalk, talc, kaolin, starch, smectite clays, chemically modified magnesium aluminum silicate, organically modified montmorillonite clay, hydrated aluminum silicate, fumed silica, aluminum starch octenyl succinate and mixtures thereof.

Other adjunct minor components may also be incorporated into the cosmetic compositions. These ingredients may include coloring agents, opacifiers and perfumes. Amounts of these other adjunct minor components may range anywhere from 0.001% up to 20% by weight of the composition.

Accordingly, a composition of the invention comprises skin lightening agents, alpha hydroxyl acids, and a blend of tea extracts in an aqueous kaolin clay vehicle. Furthermore, a composition of the invention may include additional agents or additives that are not in themselves active agents but play a role in promoting the usefulness or effectiveness of an active agent.

Compositions of the invention may be applied to any subject and used to treat a variety of conditions, particularly for the reducing the appearance of spots and discoloration, reducing the appearance of redness, brightening skin and reducing the appearance of fine lines and wrinkles. A typical composition of the invention is formulated as a mask, which may be applied topically to the skin for a period of from about 5 minutes to about 30 minutes, usually from about 10 minutes to about 15 minutes, and rinsed off with, for example water, so as to treat, prevent, condition or otherwise effect a condition of the skin.

### Product Use, Form, and Packaging

In use, a quantity of the composition, for example from 1 to 100 ml, is applied to a site of interest from a suitable container or applicator and, if necessary, it is then spread over and/or rubbed into the site using the hand or fingers or a suitable device. The product may be specifically formulated for use as a treatment for a specific area, e.g. the neck, the face, etc.

The cosmetic composition of the invention can be formulated in any form suitable for application to the site of interest, including a lotion, cream, gel, or the like. The composition can be packaged in any suitable container to suit its viscosity and intended use by the consumer. For example, a lotion or cream can be packaged in a bottle, or a propellant-driven aerosol device or a container fitted with a pump suitable for finger operation. When the composition is a cream, it can simply be stored in a non-deformable bottle or squeeze container, such as a tube or a lidded jar. The invention accordingly also provides a closed container containing a cosmetically acceptable composition as herein defined.

The following examples are put forth so as to provide those of ordinary skill in the art with a complete disclosure and description of how to make and use the subject invention, and are not intended to limit the scope of what is regarded as the invention. Efforts have been made to insure accuracy with respect to the numbers used (e.g. amounts, temperature, concentrations, etc.) but some experimental errors and deviations should be allowed for. Unless otherwise indicated, parts are parts by weight, molecular weight is weight average molecular weight, temperature is in degrees centigrade, and pressure is at or near atmospheric.

### EXAMPLE 1

Example 1 illustrates a topical composition according to the present invention. The composition can be processed in conventional manner and is suitable for cosmetic use. In particular the compositions are suitable for application to a site of interest for the treatment of a variety of skin conditions.

Marini Luminate Face Mask

| Name | CAS number | Final concentration |
|---|---|---|
| Dimethylmethoxy Chromanyl | 1105025-85-1 | 0.05-0.5% |
| Palmitate | | |
| Alpha arbutin | 84380-01-8 | 0.1%-0.5% |
| Dipotassium glycyrrhizate | 68797-35-3 | 0.1%-1% |
| Lactic acid | 79-33-4 | 2.5%-7.5% |
| Mandelic acid | 90-64-2 | 2.5%-7.5% |
| Camellia sinensis extract | 84650-60-2 | 0.1%-0.5% |
| Camellia oleifera extract | 94333-93-4 | 0.1%-0.5% |
| Aspalathus linearis extract | | 0.1%-0.5% |
| Kaolin | 1332-58-7 | 7.5%-12.5% |

Additional ingredients can be included to provide a cosmetically acceptable vehicle, and may comprise water, cyclic polysiloxanes, glycerin, caprylic/capric triglyceride, glyceryl stearate, Butyrospermum parkii (shea) butter, cetyl alcohol, dimethicone, cyclopentasiloxane, citric acid, Prunus amygdalus dulcis (sweet almond) oil, cyclohexasiloxane, Glycine soja (soybean) oil, sodium stearoyl glutamate, squalane, aluminum starch octenylsuccinate, pentylene glycol, sodium oleate, hydrogenated lecithin, ethyl alcohol, coco-glucoside, caprylyl glycol, sodium citrate, ethylhexylglycerin, hexylene glycol, acrylates/C10-30 alkyl acrylate crosspolymer, xanthan gum, triethanolamine, disodium edta, phenoxyethanol.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A cosmetic composition for topical application comprising:
from 0.05-0.5% by weight dimethylmethoxy chromanyl palmitate; from 0.1%-0.5% by weight alpha arbutin; from 0.1%-1% dipotassium glycyrrhizate; from 2.5%-7.5% by weight lactic acid; from 2.5%-7.5% by weight mandelic acid; and
a cosmetically acceptable vehicle.

2. The composition of claim 1, further comprising a blend of tea extracts, comprising from 0.1%-0.5% by weight Camellia sinensis extract; from 0.1%-0.5% by weight Camellia oleifera extract; and from 0.1%-0.5% by weight Aspalathus linearis extract.

3. The composition of claim 2, formulated as a mask.

4. The formulation of claim 3, wherein the formulation further comprises from 7.5%-12.5% by weight kaolin.

5. The composition of claim 1, comprising:
from 0.05-0.5% by weight dimethylmethoxy chromanyl palmitate; from 0.1%-0.5% by weight alpha arbutin; from 0.1%-1% dipotassium glycyrrhizate; from 2.5%-7.5% by weight lactic acid; from 2.5%-7.5% by weight mandelic acid; from 0.1%-0.5% by weight Camellia sinensis extract; from 0.1%-0.5% by weight Camellia oleifera extract; and from 0.1%-0.5% by weight Aspalathus linearis extract; and
a cosmetically acceptable vehicle comprising from 7.5%-12.5% by weight kaolin.

6. A method of improving the appearance of the skin and reducing undesirable pigmentation, comprising:
topically applying a cosmetic composition comprising:
from 0.05-0.5% by weight dimethylmethoxy chromanyl palmitate; from 0.1%-0.5% by weight alpha arbutin; from 0.1%-1% dipotassium glycyrrhizate; from 2.5%-7.5% by weight lactic acid; from 2.5%-7.5% by weight mandelic acid; and a cosmetically acceptable vehicle, for a period of from about 5 to about 30 minutes; and
rinsing free.

7. The method of claim 6, wherein the composition further comprises a blend of tea extracts, comprising from 0.1%-0.5% by weight Camellia sinensis extract; from 0.1%-0.5% by weight Camellia oleifera extract; and from 0.1%-0.5% by weight Aspalathus linearis extract.

8. The method of claim 7, wherein the composition further comprises from 7.5%-12.5% by weight kaolin.
